(19) United States
(12) Patent Application Publication
(10) Pub. No.: US 2007/0225681 A1
(43) Pub. Date: Sep. 27, 2007

(54) Catheter Having a Selectively Formable Distal Section

(75) Inventor: Morgan House, Newfields, NH (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, SANTA ROSA, CA (US)

(21) Appl. No.: 11/277,062

(22) Filed: Mar. 21, 2006

(51) Int. Cl. A61M 25/00

(52) U.S. Cl. 604/528

(57) ABSTRACT

The current invention discloses a delivery catheter with a selectively formable distal section. The catheter comprises a central lumen that is configured to receive a puncture catheter that is used for puncturing the septum of a heart and to emplace devices used for treating mitral regurgitation. The delivery catheter includes control members disposed in a control lumen, and a plurality curved areas can be selectively formed in the distal section of the delivery catheter by applying tension to the control members. A first curve is shaped to conform to the interior of a heart chamber and a combination of the first curve and a second curve allows a clinician to manipulate the distal end of the catheter for selection of the proper vector for deploying a treatment device, or for guiding a treatment device around obstacles in a heart chamber.

### TECHNICAL FIELD

This invention relates generally to medical devices and particularly to a system and method for treating mitral valve regurgitation by reducing the lateral space between the ventricular septum and the free walls of the left ventricle.

### BACKGROUND OF THE INVENTION

The heart is a four-chambered pump that moves blood efficiently through the vascular system. Blood enters the heart through the vena cava and flows into the right atrium. From the right atrium, blood flows through the tricuspid valve and into the right ventricle, which then contracts and forces blood through the pulmonic valve and into the lungs. Oxygenated blood returns from the lungs and enters the heart through the left atrium and passes through the bicuspid mitral valve into the left ventricle. The left ventricle contracts and pumps blood through the aortic valve into the aorta and to the vascular system.

The mitral valve consists of two leaflets (anterior and posterior) attached to a fibrous ring or annulus. In a healthy heart, the mitral valve leaflets overlap during contraction of the left ventricle and prevent blood from flowing back into the left atrium. However, due to various cardiac diseases, the mitral valve annulus may become distended, causing the leaflets to remain partially open during ventricular contraction and thus allowing regurgitation of blood into the left atrium. This results in reduced ejection volume from the left ventricle, causing the left ventricle to compensate with a larger stroke volume. The increased workload eventually results in dilation and hypertrophy of the left ventricle, further enlarging and distorting the shape of the mitral valve. If left untreated, the condition may result in cardiac insufficiency, ventricular failure, and death.

It is common medical practice to treat mitral valve regurgitation by valve replacement or repair. Valve replacement involves an open-heart surgical procedure in which the patient's mitral valve is removed and replaced with an artificial valve. This is a complex, invasive surgical procedure with the potential for many complications and a long recovery period.

Mitral valve repair includes a variety of procedures to repair or reshape the leaflets to improve closure of the valve during ventricular contraction. If the mitral valve annulus has become distended, a common repair procedure involves implanting an annuloplasty ring on the mitral valve annulus. The annuloplasty ring generally has a smaller diameter than the annulus, and when sutured to the annulus, the annuloplasty ring draws the annulus into a smaller configuration, bringing the mitral valve leaflets closer together and providing improved closure during ventricular contraction.

Annuloplasty rings may be rigid, flexible, or have both rigid and flexible segments. Rigid annuloplasty rings have the disadvantage of causing the mitral valve annulus to be rigid and unable to flex in response to the contractions of the ventricle, thus inhibiting the normal movement of the mitral valve that is required for it to function optimally. Flexible annuloplasty rings are frequently made of Dacron® fabric and must be sewn to the annular ring with a line of sutures. This eventually leads to scar tissue formation and loss of flexibility and function of the mitral valve. Similarly, combination rings must generally be sutured in place and also cause scar tissue formation and loss of mitral valve flexibility and function.

Annuloplasty rings have been developed that do not require suturing. U.S. Pat. No. 6,565,603 discloses a combination rigid and flexible annuloplasty ring that is inserted into the fat pad of the atrioventricular groove, which surrounds the mitral valve annulus. Although this device avoids the need for sutures, it must be placed within the atrioventricular groove with great care to prevent tissue damage to the heart.

U.S. Pat. No. 6,569,198 discloses a flexible annuloplasty ring designed to be inserted into the coronary sinus, which is located adjacent to and partially surrounds the mitral annulus. The prosthesis is shortened lengthwise within the coronary sinus to reduce the size of the mitral annulus. However, the coronary sinus in a particular individual may not wrap around the heart far enough to allow effective encircling of the mitral valve, making this treatment ineffective.

U.S. Pat. No. 6,210,432 discloses a flexible elongated device that is inserted into the coronary sinus and adapts to the shape of the coronary sinus. The device then undergoes a change that causes it to assume a reduced radius of curvature and, as a result, causes the radius of curvature of the coronary sinus and the circumference of the mitral annulus to be reduced. While likely to be effective for modest changes in the size or shape of the mitral annulus, this device may cause significant tissue compression in patients requiring a larger change in the configuration of the mitral annulus.

U.S. Patent Application Publication 2003/0105520 discloses a flexible elongated device that is inserted into the coronary sinus and anchored at each end by a self-expanding, toggle bolt-like anchor that expands and engages the inner wall of the coronary sinus. Application WO02/076284 discloses a similar flexible elongated device that is inserted into the coronary sinus. This device is anchored at the distal end by puncturing the wall of the coronary sinus, crossing the intervening cardiac tissue, and deploying the anchor against the exterior of the heart in the pericardial space. The proximal end of the elongated member is anchored against the coronary ostium, which connects the right atrium and the coronary sinus. Once anchored at each end, the length of either of the elongated devices may be adjusted to reduce the curvature of the coronary sinus and thereby change the configuration of the mitral annulus. Due to the nature of the anchors, both of these devices may cause significant damage to the coronary sinus and surrounding cardiac tissue. Also, leaving a device in the coronary sinus may result in formation and breaking off of a thrombus that may pass into the right atrium, right ventricle, and ultimately the lungs, causing a pulmonary embolism. Another disadvantage is that the coronary sinus is typically used for placement of a pacing lead, which may be precluded with the placement of the prosthesis in the coronary sinus.

U.S. Pat. No. 6,616,684 discloses a splint assembly that is positioned transverse the left ventricle to treat mitral valve leakage. In one embodiment, the assembly is delivered through the right ventricle. One end of the assembly is anchored outside the heart, resting against the outside wall of the left ventricle, while the other end is anchored within the right ventricle, against the septal wall. The heart-engaging portions of the assembly, i.e., the anchors, are essentially flat and lie snugly against their respective walls. The length of the splint assembly is either preset or is adjusted to draw the two walls of the chamber toward each other.

The splint assembly may be delivered endovascularly, which offers distinct advantages over open surgery methods. However, endovascular delivery of the splint assembly can be a complicated process that involves multiple delivery steps and devices, and requiring that special care be taken to avoid damage to the pericardium and lungs. First, a needle or guidewire is delivered into the right ventricle, advanced through the septal wall, and anchored to the outer or free wall of the left ventricle using barbs or threads that are rotated into the tissue of the free wall.

Visualization is required to ensure the needle does not cause damage beyond the free wall. A delivery catheter is then advanced over the needle, piercing both the septal wall and the free wall of the ventricle. The splint assembly is then installed and the tension is adjusted to reduce mitral regurgitation. One problem that can be associated with this procedure is that a delivery catheter may not provide sufficient support when the needle and/or or guide wire is being used to puncture the septum and then the free wall of the heart chamber. This can result in the splint being implanted at a less than optimal vector, which ultimately results in less than optimal reduction of the mitral regurgitation.

Additionally, a clinician may have to aggressively manipulate the delivery device to provide sufficient support to puncture the septum. Thus, there is an increased risk of causing injury or damage to the interior structure of the right ventricle and/or atrium. This risk also exists where the septum must be punctured between the right atrium and left atrium. Thus, there is a need for a catheter having a distal section that can be delivered through a patient's vasculature, to a chamber in the patient's heart, whereupon the distal section of the catheter can be manipulated to conform to the interior of the heart chamber such that the catheter provides stable support for delivering devices used to treat heart valves.

Some catheters and endoscopes can be remotely steered. For example, U.S. Pat. No. 5,325,845 suggests a steerable sheath for use in connection with optical catheters. The proximal end of the catheter is provided with a pair of steering knobs which are connected to wires that run along the length of the catheter. Each knob controls a pair of diametrically opposed wires and all four of the wires are attached to the distal tip of the catheter. By appropriate manipulation of either of the control knobs, one can ostensibly control the position of the distal tip of the catheter. By such remote manipulation, the reference claims a physician can move the optical catheter into position to view the desired site. Others have proposed similar uses of cables in endoscopic procedures. For example, U.S. Pat. No. 4,700,693 suggests a design which utilizes steering cables and a number of washers. The steering cables can be remotely manipulated to guide the endoscope through a desired curve.

There are also steerable and formable catheters that can be used to deliver therapeutic devices to a body through lumens in the catheter. U.S. Pat. No. 5,916,147, U.S. Pat. No. 6,544,215, and U.S. Pat. No. 6,991,616 are examples of such catheters. Also, the use of catheters for puncturing the septum in the right atrium is well known in the art, but those catheters can not be formed to provide the stability and support required for delivering devices used to treat heart valves. Thus, there have been no catheters available that can proved adequate stability and support to devices that can be used to puncture a septum and then deliver other devices for treating heart valves.

Therefore, it would be desirable to provide devices that can provide adequate stability and support to catheters and other devices being used to puncture a septum in a heart. Such devices would allow clinicians more control in the location of a puncture in a septum and the placement location of devices for treating heart valves. While there are several patents that discloses

### SUMMARY OF THE INVENTION

The present invention discloses a catheter having a selectively formable distal section that can be used as a delivery catheter for a septal puncture and heart valve treatment system. The selectively formable distal section comprises a first curve and a second curve that can be selectively formed by applying tension to a first and second control member. The control members are disposed in a control member lumen and they extend from openings in the distal region of the lumen to a more distal point, where each is affixed to the catheter. Tension is applied to the control members by manipulating adjustment members on the proximal portion of the catheter.

Each curve has an apex and a base, with a control member extending across and defining the base of the curve section. The first curve is formed to have a shape that corresponds to the interior shape of a heart chamber so that the catheter can be braced against the interior wall of the heart chamber. The combination of the curve being braced against the wall and the control member extending across the base of the curve provides a stable support for use when extending the puncture system through the septum.

The two curves operate in generally perpendicular planes, which along with center axis rotation and longitudinal motion provide the capability to direct the distal end of the catheter in a wide range of directions such that the puncture system can extend from the delivery catheter in a desired vector. The curves also allow for a wide range of motion at the distal tip of the catheter for maneuvering the puncture system and treatment systems around obstacles in the heart chamber.

In another embodiment of the current invention, the catheter has a slot that communicates from the central lumen to the catheter exterior such that the puncture system can exit the catheter. The slot starts at a location distal to a point on the catheter having a first opening in a control member lumen and extends distally to a location that is proximal to a first attachment point. When the catheter is manipulated to form a first curve, the puncture system can separate from the central lumen for some of the distance along the curved portion and extend across the curve and back into the central lumen. This configuration allows for delivery of the puncture system with out the need for the system to follow the tortuous path of the curve.

Another aspect of the current invention is that it discloses a method for puncturing the septum in a heart. To perform the method, a clinician must first determine the size and shape of a heart chamber. A catheter is then selected based on the shape, and the distal end of the catheter is navigated through the vasculature to the heart chamber. Control members for the catheter are then manipulated to selectively form a first curve in the catheter, whereby the first curve has a shape that corresponds to the interior shape of the heart chamber and is supported against the walls of the heart chamber. A second curve is then formed in the catheter to direct the distal tip of the catheter in a desired direction. A puncture system is then extended from the distal tip of the catheter and through the septum so that a system for treating diseased heart valves can be deployed.

Catheters disclosed herein are advantageous over previously disclosed devices in that they provide a firm stable support for bracing a catheter in a heart chamber while a puncture system is used to puncture the septum. The catheters disclosed herein also allow a clinician to select from a wide range of possible directions so that treatment systems can be properly deployed. The wide range of motion also allows the distal tip of the catheter to be manipulated to guide the puncture system and other devices around obstacles in the heart.

The aforementioned and other features and advantages of the invention will become further apparent from the following detailed description of the presently preferred embodiments, read in conjunction with the accompanying drawings, which are not to scale. The detailed description and drawings are merely illustrative of the invention rather than limiting, the scope of the invention being defined by the appended claims and equivalents thereof.

### BRIEF DESCRIPTION OF THE DRAWINGS

FIG. 1 shows a catheter having a selectively formable distal section according to the current invention.

FIG. 2 shows an enlarged view of the distal section of a catheter according to the current invention.

FIG. 3 shows a cross-section of one embodiment of a catheter according to the current invention.

FIG. 4 is a cross-sectional schematic view of a heart showing the placement of a catheter through the tricuspid valve in the right atrium according to the current invention.

FIG. 5 is a cross-sectional schematic view of a heart showing the placement of a catheter in the right ventricle according to the current invention.

FIG. 6 is a cross-sectional schematic view of a system for puncturing the septum in a heart and a device for treating mitral regurgitation according to the current invention.

FIG. 7 is a schematic view illustrating the positioning of a catheter in the right ventricle and the puncturing of the septum according to the current invention.

FIGS. 8 & 9 show an alternate preferred embodiment of a catheter having a selectively formable distal section according to the current invention.

### DESCRIPTION OF THE PREFERRED EMBODIMENT

The invention will now be described in detail below by referring to the attached drawings, where like numbers refer to like structures. One aspect of the present invention is a catheter having a selectively formable distal portion. Curves having shapes that correspond to the interior anatomy of the chambers of the heart can be formed by manipulating control members of the catheter. The catheter can be braced against the chamber walls during deployment of devices used for treating valvular diseases.

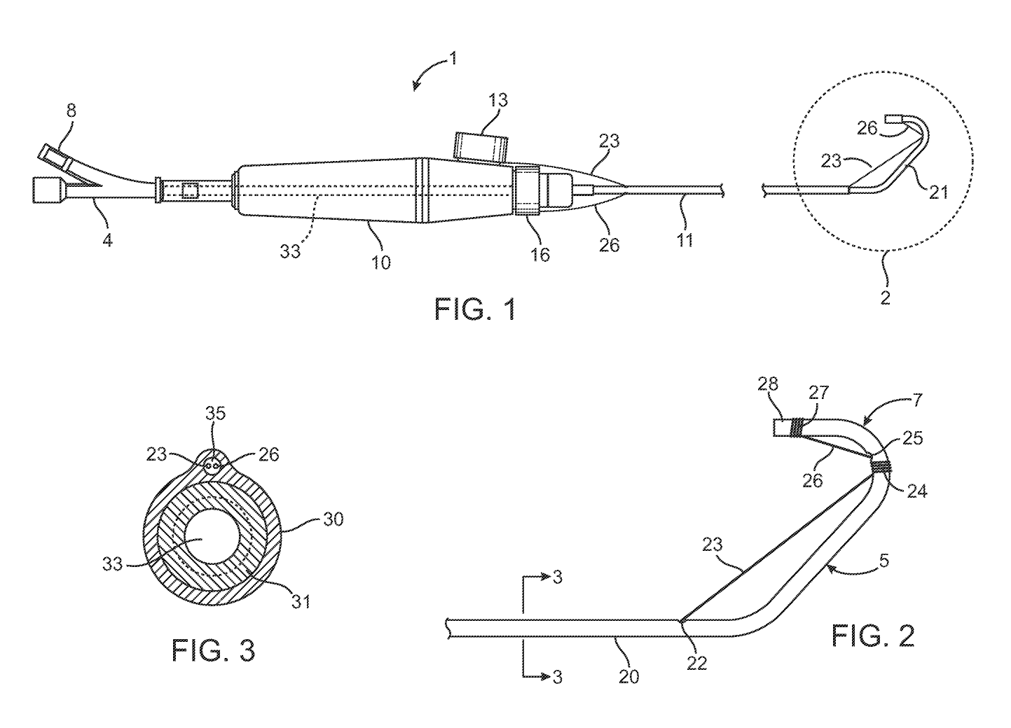

Referring to FIG. 1, there can be seen a catheter having a selectively formable distal tip according to the current invention. The catheter 1 comprises a handle 10, a proximal section 11, and a distal section 21. As used herein, the term proximal means the portion or end of the catheter that is closest to the clinician manipulating the catheter when it is in use and distal means that portion or end of the catheter that is further away from the clinician when the catheter is in use. The proximal section of the catheter is the portion that is forward or distal of the handle but proximal of the midpoint of the catheter and the distal section of the catheter is that portion that is distal of the proximal section.

Referring now to FIG. 2, there can be seen an enlarged view of the portion of the distal section of the catheter that is shown inside of the dotted line circle 2 of FIG. 1, and FIG. 3 shows a cross-section of the catheter taken at line 3-3 of FIG. 2. Now referring to FIGS. 1-3, a preferred embodiment of a catheter 1 according to the current invention has a handle 10 having an input port 4 and an injection port 8. A lumen 33 runs through the handle and along the length of the catheter through the proximal section 11 and the distal section 21 before terminating in an opening at the distal tip 28. The lumen 33 can be used for delivering septal puncture systems or systems for treating valvular disease to the chambers of a heart.

The wall of the catheter of the depicted embodiment can include a reinforced layer of biocompatible material 31. The material can be any material known by those having ordinary skill in the art to be suitable for constructing catheters, including PEBAX. The pictured embodiment includes reinforcement in the catheter walls 31, and the reinforcement can be any material known by those having ordinary skill in the art to be suitable for catheter construction, including braided metal or alloy fibers. While it may not be included in every embodiment of the invention, the depicted embodiment includes an outer jacket 30 having a control member lumen 35. The outer jacket can be made from flexible, biocompatible polymeric materials that are suitable for catheter construction and examples of such material include, but are not limited to, polyurethane, polyethylene, nylon and polytetrafluoroethylene (PTFE).

A first control member 23, and a second control member 26 are disposed in the control member lumen. The proximal end of the first control member 23 extends from an opening in control member lumen that is located in the proximal section 11 of the catheter and it is connected to a first adjustment member 13. The proximal end of the second control member 26 extends from an opening in control member lumen that is located in the proximal section 11 of the catheter and it is connected to a second adjustment member 16. the control members of the depicted embodiment can be made from any appropriate biocompatible material including line made from braiding polymeric fibers, and in one preferred embodiment the line is made from polyethylene fibers. Other materials are also suitable for making the control members including braided and single strand metal wires.

Referring to FIG. 2, the control member lumen includes a first opening 22 that is distal of the midpoint of the catheter, but proximal of the distal tip 28 of the catheter. The first control member 23 extends distally from the opening and it is affixed to the catheter at an anchor point 24 that is distal of the first opening and proximal of the distal tip 28. The second control member 26 extends from a second opening 25 in the control member lumen that is distal of the first opening 22. The second control member is affixed to the catheter at a second anchor point 27 that is distal of the second opening 25.

The control member lumen in one preferred embodiment of the current invention extends along one side of the catheter without winding around the catheter. The control member lumen in another preferred embodiment winds around the central lumen of the catheter, so that the control member openings do not have to be on the same side of the catheter. Allowing the control member lumen to wind around the central lumen allows some flexibility in selecting locations for the control member openings to provide for curved sections with other desired shapes and to provide for alternate desired vectors for emplacing treatment devices. Yet another preferred embodiment includes more than one control member lumens, each having at least one control member opening in the distal section. In another preferred embodiment, the control member lumen/lumens do not have openings in the distal section and the control members do not exit the control member lumen/lumens.

The distal section 21 of the depicted catheter is selectively formable into a first curve 5 by selectively manipulating the first adjustment member 13 to apply tension to the first control member 23 such that the first anchor point 24 is drawn toward the first opening 22 and a first curve 5 is formed. The first control member defines the base of the curve by spanning the space between the first anchor point and the first opening. A second curve 7 can be formed by selectively manipulating the second adjustment member 16 to apply tension to the second control member 26 such that the second anchor point 27 is drawn toward the second opening 25 and a second curve 7 is formed. The second control member defines the base of the curve by spanning the space between the second anchor point and the second opening.

In the depicted embodiment, tension is applied by rotating the adjustment members, which causes the control member to wind around a base of the adjustment member. Other embodiments of the invention can use other methods of operation for the adjustment members while applying tension to the control members.

Those having ordinary skill will understand that catheters having suitable flexibility for practicing the invention disclosed herein can be made to have different materials along the length of the catheter, and/or different thicknesses of the catheter walls along the length to achieve the desired flexibility, handling characteristics, and kink resistance. The catheters can include outer jacket layers, layers of braided material, or have fibers of reinforcing material integrally formed therein. Other structures and techniques can also be used to form catheters having suitable characteristics for practicing the disclosed invention.

In one preferred embodiment, the first curve has a shape that is complementary to the inside shape of the right ventricle of a heart so that the catheter can be braced against opposing walls inside the chamber while the septum is being punctured and devices for treating diseased heart valves are being delivered to the heart tissue. In another embodiment, the first curve has a shape that is complementary to the interior of the right atrium such that the curve can be braced against opposing heart walls above the tricuspid valve annulus while the septum is being punctured and devices for treating diseased heart valves are being delivered to the heart tissue. The second control member is manipulated to selectively form the second curve so that the distal tip is oriented such that devices for treating diseased heart valves can be deployed in the proper direction relative to the catheter. The control members extend across the base of the curves to provide additional stability and support when treatment devices are being deployed from the catheter.

While the embodiment depicted in the figures is configured for having two selectively formable curves, other embodiments can include more selectively formable curves, and the curves will be formed in a manner similar to that described above. Additionally, other embodiments of the invention can include catheters with a round or oval shaped cross-section and no exterior jacket/layer.

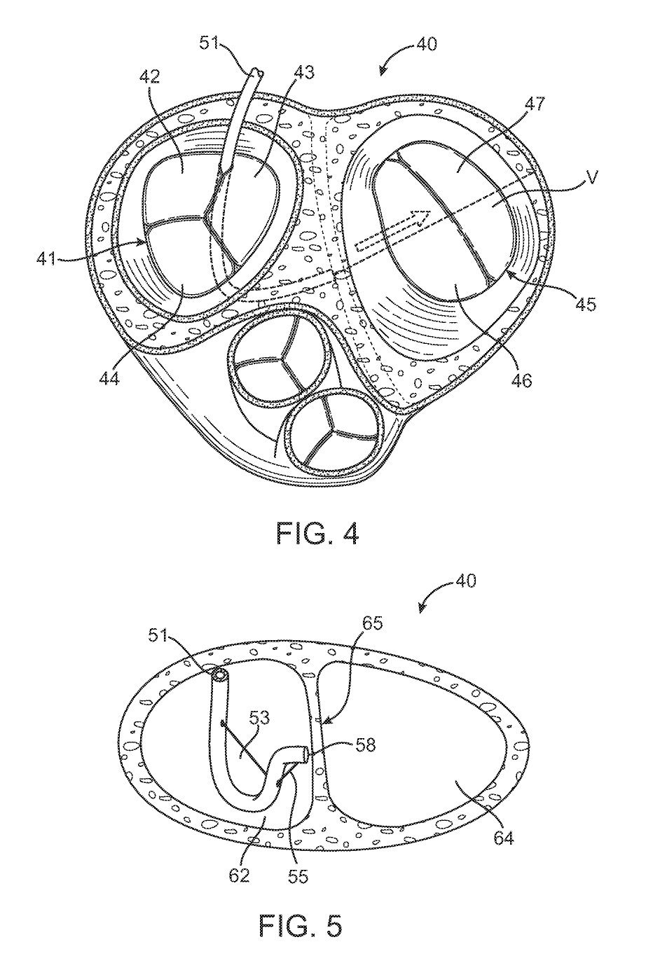

FIG. 4 is a cross-sectional view of a heart 40 having a tricuspid valve 41 with a posterior leaflet 42, an anterior leaflet 44, and a septal leaflet 43. The heart also includes a mitral valve 45 having an anterior leaflet 46 and a posterior leaflet 47. A catheter 51 of the current invention passes through the right atrium of the heart between the posterior leaflet 42 and the septal leaflet 43 of the tricuspid valve 41.

FIG. 5 is a cross-sectional view of the heart 40 of FIG. 4 taken below the mitral valve and tricuspid valve annulus. The heart includes a right ventricle 62 and a left ventricle 64 that are separated by a septum 65. After the catheter 51 is routed into the right ventricle, tension can be applied to a first control member 53 to selectively form a curve such that the catheter is braced against opposing interior walls of the heart chamber, with one side of the curve braced against the free wall of the ventricle and the other side braced against the septum. Tension can then be applied to the second control member 55 to manipulate the distal tip 58 such that devices can be deployed along the correct vector. In one procedure, a device is deployed in the left ventricle such that it extends across the ventricle on a vector (shown as V in FIG. 4) that is generally perpendicular to the edges of the leaflets of a mitral valve. Tension is applied to the device, and the leaflets are drawn closer together. Devices and methods for treating mitral regurgitation are disclosed in U.S. patent applications Ser. Nos. 10/531,819 and 10/867,394, the contents of both being incorporated herein by reference.

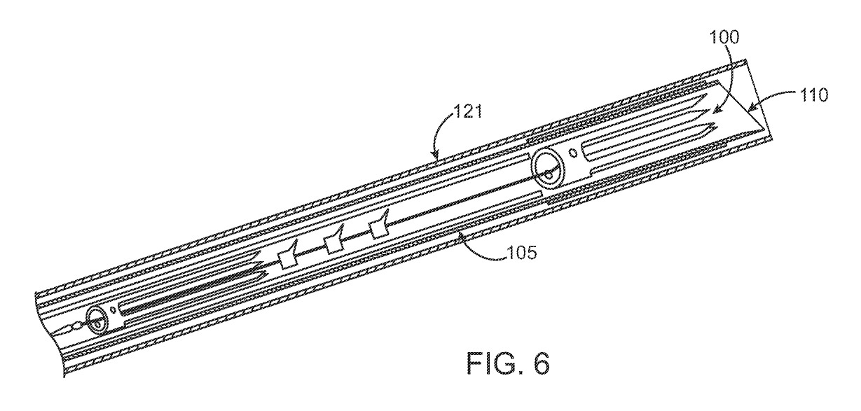

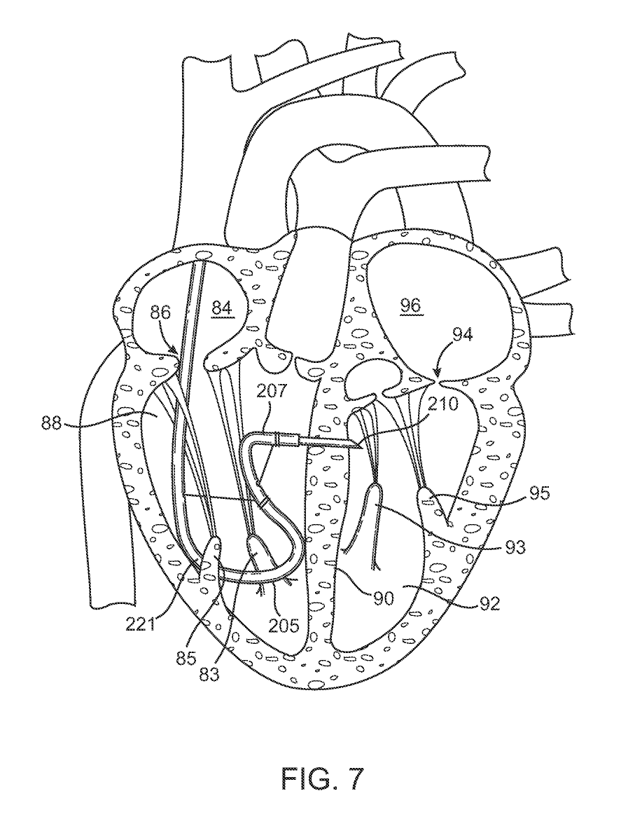

FIG. 6 shows an embodiment of a catheter with a selectively formable distal section 121 having a device used for treating mitral regurgitation according to the current invention. The device 100 is configured for placement across the left ventricle of a heart. The device 100 is disposed in a puncture catheter 105 having a sharpened distal tip 110. The puncture device and the tension device can be made from any suitable biocompatible material. In one embodiment, the puncture device is a hypo tube.

FIG. 7 shows a catheter 221 according to the current invention being used to deliver a puncture device 210 for puncturing the septum 90. The catheter is passed through the right atrium 84 and the tricuspid valve 86. Tension is then applied to a first control member to form a curve 205 at the distal end of the catheter such that the catheter is braced against the walls of the right ventricle 88, such that it rests against the septum and the opposite free wall of the heart chamber. Tension is then applied to a second control member to form a curve 207 such that the distal tip of the catheter is oriented in the proper direction. The puncture device 210 is then advanced through the septum 90 and into the left ventricle 92. A device for treating regurgitation of the mitral valve 94 can then be secured in the ventricle. The distal tip of the catheter can also be manipulated to avoid the chordae and other critical structure by selectively applying tension to the second control member.

The catheter is delivered to the heart by passing it through the venous system. This may be accomplished by inserting the catheters into either the jugular vein or the subclavian vein and passing it through the superior vena cava and into the right atrium. Alternatively, the catheter may be inserted into the femoral vein and passed through the common iliac vein and the inferior vena cava into the right atrium. Catheters of the current invention can be delivered through the vasculature to the heart using over-the-guidewire techniques, or they can be delivered without the use of a guidewire.

Embodiments of the catheters, puncture devices, and treatment devices disclosed or discussed herein can include materials having a high X-Ray attenuation coefficient (radiopaque material) such that the procedure may be visualized. The material can be placed or located on the devices in a manner that would be readily apparent to one of ordinary skill in the art. In one embodiment of the current invention, the catheter and the puncture device each have bands of radiopaque material spaced along a portion of the distal sections thereof, and the treatment device has radiopaque material disposed thereon. Other parts of the catheters and devices that would be useful to see during the procedures can also be coated with radiopaque material. Examples of suitable radiopaque material include, but are not limited to gold, tungsten, silver, iridium, platinum, barium sulfate and bismuth sub-carbonate. The procedure may be visualized using fluoroscopy, echocardiography, intravascular ultrasound, angioscopy, or other means of visualization.

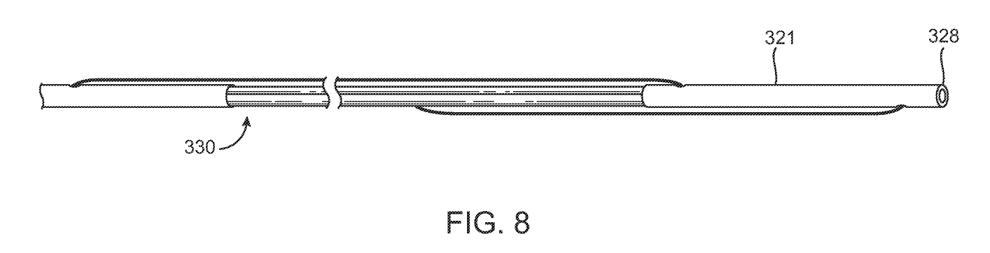

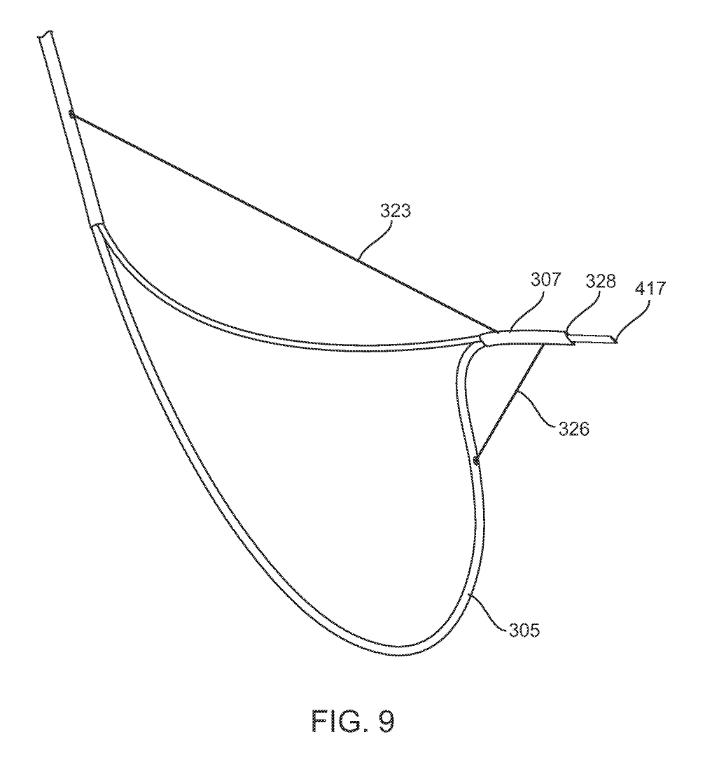

FIG. 8 and FIG. 9 show an embodiment of the invention where a portion of the distal section of the catheter 321 includes a slot or cut away area 330 that communicates from the outside of the catheter into the central lumen. This embodiment allows the puncture device 410 to take a less tortuous path to the distal tip of the catheter. When tension is applied to the first control member 323, and a first curve 305 is formed, the puncture device exits the catheter at the proximal end of the slotted portion, and renters the catheter at the distal end of the slotted portion. Tension can be applied to the second control member to form a second curve for pointing the distal tip of the catheter in the desired direction. The first curve can brace against the inside walls of a heart chamber and provide support and stability for puncturing the septum of the heart. The control member spanning the base of the curve also provides support during puncture procedures and while emplacing devices for treating diseased heart valves.

The current invention discloses embodiments of catheters having a distal section that is configured for selective formation into at least one curve that has a shape complementary to the interior of the chambers of a heart. The distal section of one embodiment can be selectively formed into a curve having a shape complementary to the interior of a right ventricle, and the distal segment of another embodiment can be formed into a curve having a shape that is complementary to the right atrium. One embodiment of the catheters depicted herein has two selectively formable curves in the distal section, and another embodiment has more than two selectively formable curve sections.

One embodiment of a catheter according to the current invention can include at least three control members, and at least three selectively formable curves such that curves having shapes that are complementary to the interior of either the right atrium or the right ventricle can be formed in the distal section of the catheter based on the operator's discretion and the procedure being performed.

To perform procedures using the catheters disclosed herein, a clinician can navigate to the selected heart chamber through the vasculature in the manner described above. Once the distal section of the catheter is in the selected heart chamber, tension is applied to the first control member via the first adjustment member to form a first curve in the distal section, wherein the first curve has a shape that will allow the catheter to brace against the heart walls for stability and support. A second curve is then formed by applying tension to the second control member. The direction that the distal tip is facing can then be adjusted by adjusting the tension on the first and second control members. Once the distal tip of the catheter is properly oriented, a puncture device can be extended from the end of the catheter to puncture the septum, and a device for treating diseased heart valves can then be installed. The combination of the catheter bracing against the walls of the heart chamber, and the control member extending across the base of the formed curves, provides stable support for the puncture of the septum and installation of the device.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A catheter for use in a medical procedure comprising:
an elongate, flexible, generally tubular member having a proximal section, a distal section, a distal tip an exterior surface, and a central lumen;
a control member lumen extending along a majority of the length of the elongate member;
at least at least two elongate, flexible control members, each having a distal end that is affixed to the elongate tubular member and a proximal end, and each control member being disposed in the control member lumen;
the control lumen having at least two openings and at least two attachment points spaced along the distal section thereof, and one control member extends distally from each opening to one of the attachment points; and
the distal section of the catheter being selectively formable into at least two curves by applying tension to the control members such that one curve can be formed for each control member.

2. The catheter of claim 1 wherein the central lumen communicates from an opening in the proximal section of the elongate member, through a majority of the length of the elongate member, and terminates in an opening at the distal tip of the elongate member;

3. The catheter of claim 1 wherein the at least two control members is first control member and a second control member;
the at least two openings in the control member lumen is a first opening that communicates from the lumen to the exterior of the elongate member at a location proximal of the distal tip of the elongate member and a second opening that communicates from the lumen to the exterior of the elongate member at a location distal of the first opening; and
the at least two attachment points is a first attachment point that is distal of the first opening and a second attachment point that is distal of the second opening.

4. The catheter of claim 1 wherein the proximal ends of the at least two control members are each connected to an adjustment member that is operably attached to the proximal section of the elongate member; and
the adjustment member can be manipulated to apply tension to the control member such that a curve is selectively formed in the distal section of the catheter.

5. The catheter of claim 4 wherein the control member lumen has a proximal opening along the proximal section of the catheter, and the control members extend from the proximal opening to the adjustment member.

6. The catheter of claim 1 wherein the control members are selected from the group consisting of wire, woven metallic cable, non-metallic cord.

7. The catheter of claim 1 wherein the shape of one of the at least two curves is complementary to the interior shape of a chamber of a heart such that the catheter can be braced against the walls of a heart chamber on opposite sides of the chamber.

8. The catheter of claim 1 wherein when the curves are selectively formed in the distal section of the catheter, the directional orientation of the distal end can be manipulated by adjusting tension on the control members.

9. The catheter of claim 1 wherein the at least two elongate, flexible control members comprises more than two control members;
the at least two openings comprises more than two openings;
the at least two attachment points comprises more than two attachment points; and
the at least two curves comprises more than two curves.

10. The catheter of claim 1 wherein the central lumen is configured to receive devices for puncturing the septum in a heart and devices for treating heart valves therein.

11. An apparatus for performing a medical procedure comprising:
an elongate, flexible catheter having a proximal section, a central lumen that communicates from an opening in a proximal section to an opening in the distal tip, a control member lumen, at least two control members, a distal tip, and a distal section that can be selectively formed into at least two curves each having a control line extending from an opening in the control line lumen and across the curve to an attachment point on the catheter; and
a device, disposed in the central lumen of the catheter, for puncturing the septum between two chambers of a heart.

12. The apparatus of claim 11 further comprising a device, disposed in the catheter, for providing therapy to a cardiac valve.

13. The catheter of claim 11 wherein the central lumen communicates from an opening in the proximal section of the catheter, through a majority of the length of the catheter, and terminates in an opening at the distal tip of the catheter;

14. The catheter of claim 11 wherein the at least two control members is first control member and a second control member and the catheter further comprises;
a first opening that communicates from the lumen to the exterior of the elongate member at a location proximal of the distal tip of the elongate member and a second opening that communicates from the lumen to the exterior of the elongate member at a location distal of the first opening; and
a first attachment point that is distal of the first opening and a second attachment point that is distal of the second opening.

15. The catheter of claim 11 wherein the proximal ends of the at least two control members are each connected to an adjustment member that is operably attached to the proximal section of the elongate member; and
the adjustment member can be manipulated to apply tension to the control member such that a curve is selectively formed in the distal section of the catheter.

16. The catheter of claim 11 wherein the shape of one of the at least two curves is complementary to the interior shape of a chamber such that the catheter can be braced against the walls of a heart chamber on opposite sides of the chamber.

17. A catheter for use in a medical procedure comprising:
an elongate, flexible, generally tubular member having a proximal section, a distal section, a distal end and an exterior surface;
a central lumen that communicates from an opening in the proximal section of the elongate member, through a majority of the length of the elongate member, and terminates in an opening at the distal end of the elongate member;
a control member lumen extending along a majority of the length of the elongate member;
at least a first control member and a second control member, the control members each being flexible, each having a distal end that is affixed to the elongate tubular member and a proximal end, and each control member being disposed in the control member lumen;
the control lumen having a first opening that communicates from the lumen to the exterior of the elongate member at a location in the distal section of the elongate member, but proximal of the distal end of the elongate member;
the first control member extending out of the first opening in the control member lumen and distally along the elongate member, and being affixed to the elongate member at a first attachment point, which is proximal of the distal end of the elongate member;
the control lumen having a second opening that communicates from the lumen to the exterior of the elongate member at a location distal of the first opening, but proximal of the distal end of the member;
the second control member extending out of the second opening in the control member lumen and distally along the elongate member, and being affixed to the elongate member at a second attachment point, which is distal of the first attachment point; and
the proximal ends of the control members each being connected to an adjustment device that is positioned along the proximal section of the elongate member.

18. The catheter of claim 17 wherein the first and second control members are selected from the group consisting of wire, woven metallic cable, non-metallic cord.

19. The catheter of claim 17 wherein the control member lumen has a proximal opening along the proximal section of the catheter, and the control members extend from the proximal opening to the adjustment device.

20. The catheter of claim 17 wherein the proximal end of the first control member is connected to a first adjustment device and the proximal end of the second control member is connected to a second adjustment device;
the first adjustment device being capable of manipulation to apply tension to the first control member sufficient to draw the distal end of the control member toward the first opening in the control lumen such that a first curve is formed in the catheter between the fist opening and the first attachment point;
the second adjustment device being capable of manipulation to apply tension to the second control member sufficient to draw the distal end of the control member toward the second opening in the control lumen such that a second curve is formed in the catheter between the second opening and the second attachment point; and
the first and second curves each having a base being defined by the control member.

21. The catheter of claim 20 wherein the shape of the first curve is complementary to the interior shape of a chamber of a heart such that the catheter can be braced against the walls of a heart chamber on opposite sides of the chamber.

22. A method for providing treatment to a diseased heart valve comprising the steps of:
providing an elongate, flexible catheter having a proximal section, a central lumen that communicates from an opening in a proximal section to an opening in the distal tip, a control member lumen, at least two control members, a distal tip, and a distal section that can be selectively formed into at least two curves each having a control line extending from an opening in the control line lumen and across the curve to an attachment point on the catheter;
inserting the distal end of the catheter into the vasculature of a patient;
navigating the distal end of the catheter into one of the chambers of the patient's heart;
manipulating the catheter to form a first curve having a shape that is complementary to the interior of the heart chamber such that the catheter is braced against opposite walls of the heart chamber; and
providing therapeutic treatment to the heart.

23. The method of claim 22 further comprising the steps of:
providing a device, disposed in the central lumen of the catheter, for puncturing the septum between two chambers of a heart;
providing a device, disposed in the catheter, for reducing mitral regurgitation manipulating the catheter to form a second curve in the distal section of the catheter;
manipulating the first curve and the second curve to place the distal end of the catheter in a desired orientation;
extending the device for puncturing the septum from the distal end of the catheter such that the septum is punctured;
delivering the device for reducing mitral regurgitation into the heart; and
securing the device for reducing mitral regurgitation at a pre-selected location in the heart.
